(19) United States
(12) Patent Application Publication
(10) Pub. No.: US 2018/0104063 A1
(43) Pub. Date: Apr. 19, 2018

(54) STRUCTURE FOR FACILITATING BONE ATTACHMENT

(71) Applicant: SpineCraft, LLC, Westmont, IL (US)

(72) Inventor: Wagdy W. Asaad, Burr Ridge, IL (US)

(21) Appl. No.: 15/296,802

(22) Filed: Oct. 18, 2016

(51) Int. Cl. A61F 2/30; A61F 2/44

(52) U.S. Cl. CPC A61F 2/30771; A61F 2/447; A61F 2002/4475; A61F 2002/30838; A61F 2002/30784

(57) ABSTRACT

A structure for facilitating bone attachment includes a surface and bone ingrowth features formed in the surface. Each of the bone ingrowth features comprises an opening that opens to the surface and a body that extends from the opening into the structure. The opening has a first cross-sectional dimension and the body has a second cross-sectional dimension. The second cross-sectional dimension is greater than the first cross-sectional dimension.

### FIELD OF THE INVENTION

The present invention relates to the field of surgical implant devices, more particularly to implant devices designed to encourage bone ingrowth for fusing the implant to the bone after implantation.

### BACKGROUND OF THE INVENTION

Surgical implants such as for use in the spine, knees, hips, shoulders, elbows, wrists, ankles fingers, toes, long bones and other bone structures are typically designed to promote fusion with the bone or joint into which the implant is implanted. One of the preferred methods of achieving a robust fusion is to encourage bone ingrowth into the implant itself, such as by the provision to the implant of a porous contact surface and or osteogenic coatings or particles.

Operative techniques for fusing an unstable portion of the spine or immobilizing a painful vertebral motion segment have been used for some time now. Because of the high failure rates associated with early fusion procedures using bone graft or posterior pedicle screws, different approaches to disk height maintenance using a structural graft were developed.

The Ray Threaded Fusion Cage (Stryker Spine, Allendale N.J.) is a second generation interbody fusion device for placement in the disk space between two adjacent vertebrae of the spine. The Ray Threaded Fusion Cage is a cylindrical, hollow, titanium, threaded device that screws into position within the disk space. The experience with this device is that it does not form a high level of fusion and is not mechanically stable. The contact between the cage and the opposing vertebrae is minimal, forming effectively only one line of contact along each of the opposing vertebrae. As a result, a lot of micro motion occurs between the cage and the contacted vertebrae during movements by the patient such as left to right turning, bending, etc. which effectively prevents any long lasting, permanent fusion to occur. However, used of the Ray Threaded Fusion Cage did produce relatively pain-free results in the patients into which it was implanted, as they were sufficiently stable so as not to cause pain.

The Brantigan device, also known as the Jaguar I/F Cage (DePuy Spine) can be made from titanium, PEEK (polyetheretherketone) or carbon fiber and PEEK. It can be machined to meet size and shape requirements and has achieved a high level of fusion after implantation, but has never achieved a high level of bone ingrowth, as there is generally observed a space or zone around the cage where no bone is present, although the cage has fused with the end plates.

There is a continuing need for bone implant devices in general, and particularly for interbody fusion devices that encourage bone ingrowth to the device while establishing fusion.

### SUMMARY OF THE INVENTION

According to one aspect of the present invention, a surgical implant is provided that includes: a main body having top, bottom and side surfaces; and bone ingrowth features formed in a least one of the top, bottom and side surfaces; wherein each of the bone ingrowth features comprises an opening that opens to said at least one of the top, bottom and side surfaces, and a body that extends from the opening into the implant; wherein the opening has a first cross-sectional dimension and the body has a second cross-sectional dimension; and wherein the second cross-sectional dimension is greater than the first cross-sectional dimension.

In at least one embodiment, the opening has a first cross sectional area and the body has a second cross-sectional area; and the second cross-sectional area is greater than the first cross-sectional area.

In at least one embodiment, the side surfaces are smooth.

In at least one embodiment, the bone ingrowth features are mushroom-shaped.

In at least one embodiment, the bone ingrowth features are conical-shaped.

In at least one embodiment, the bone ingrowth features are formed and shaped like trabecular bone structure.

In at least one embodiment, the bone ingrowth features are produced by 3D printing from a scanned image of trabecular bone.

In at least one embodiment, the opening has a first diameter and the body has a second diameter, the second diameter being greater than the first diameter.

In at least one embodiment, the first diameter comprises a value in a range from about 50 μm to about 600 μm and the second diameter comprises a value in a range from about 100 μm to about 1.2 mm.

In at least one embodiment, the main body comprises titanium.

In at least one embodiment, the main body comprises PEEK.

In at least one embodiment, the surgical implant comprises an interbody fusion implant.

In at least one embodiment, the surgical implant is produced by 3D printing.

In at least one embodiment, the surgical implant is produced by direct metal laser sintering.

In another aspect of the present invention, a structure for facilitating bone attachment comprising: a structure comprising a surface; and bone ingrowth features formed in said structure; wherein the bone ingrowth features comprise openings that open to the surface, and bodies that extend from the openings into the structure; wherein the openings have first cross-sectional dimensions and the bodies have second cross-sectional dimensions; and wherein at least one of the second cross-sectional dimensions is greater than at least one of the first cross-sectional dimensions from which said bodies extend, respectively.

In at least one embodiment, at least one of said openings has a first cross sectional area and at least one of said bodies that extends from said at least one of said openings, respectively, has a second cross-sectional area; and the second cross-sectional area is greater than the first cross-sectional area.

In at least one embodiment, the surface is smooth.

In at least one embodiment, the bone ingrowth features are mushroom-shaped.

In at least one embodiment, the bone ingrowth features are conical-shaped.

In at least one embodiment, at least one of said openings has a first diameter and the at least one of said bodies that extends from said at least one of said openings, respectively, has a second diameter, the second diameter being greater than the first diameter.

In at least one embodiment, the first diameter comprises a value in a range from about 50 μm to about 600 μm and the second diameter comprises a value in a range from about 100 μm to about 1.2 mm.

In at least one embodiment, the structure is produced by 3D printing.

In at least one embodiment, the structure is produced by direct metal laser sintering.

In another aspect of the present invention, a structure for facilitating bone attachment includes: a structure having a surface; and bone ingrowth features formed in the structure; wherein the bone ingrowth features are formed and shaped like trabecular bone structure; and wherein the bone ingrowth features are produced by 3D printing from a scanned image of trabecular bone.

In at least one embodiment, at least one of the bone ingrowth features comprises an opening that opens to the surface, and a body that extends from the opening into the structure; wherein the opening has a first cross-sectional dimension and the body has a second cross-sectional dimension; and wherein the second cross-sectional dimension is greater than the first cross-sectional dimension.

In another aspect of the present invention, a method of making a structure for provide an image of lattice structure of the trabecular bone; processing the scan to form a computer image model of the lattice structure; and forming the lattice structure on a surface, using a 3D printing technique, the forming performed layer-by-layer to reproduce the 3D structure of the lattice structure of the trabecular bone.

In at least one embodiment, the scan is performed by using a micro-computer tomography (micro-CT) scanner.

In at least one embodiment, the 3D structure comprises titanium.

In at least one embodiment, the 3D structure comprises PEEK.

These and other features of the invention will become apparent to those persons skilled in the art upon reading the details of the products and methods as more fully described below.

### BRIEF DESCRIPTION OF THE DRAWINGS

In the course of the detailed description to follow, reference will be made to the attached drawings. These drawings show different aspects of the present invention an, where appropriate, reference numerals illustrating like structures, components, materials and/or elements in different figures are labeled similarly. It is understood that various combinations of the structures, components, materials and/or elements, other than those specifically shown, are contemplated and are within the scope of the present invention.

FIG. 1 shows a perspective view of an implant according to an embodiment of the present invention.

FIG. 2 shows a top view of the implant of FIG. 1.

FIG. 3 is a partial, longitudinal sectional view of the implant of FIG. 2 taken along line A-A.

FIG. 4 is a partial, longitudinal sectional view of the implant of FIG. 2, taken along line A-A, according to another embodiment of the present invention.

FIG. 5 is a partial, longitudinal sectional view of the implant of FIG. 2, taken along line A-A, according to another embodiment of the present invention.

FIG. 6 is a partial, longitudinal sectional view of the implant of FIG. 2, taken along line A-A, according to another embodiment of the present invention.

FIG. 7 illustrates an implant employing radiopaque markers, according to an embodiment of the present invention.

FIG. 8 shows a perspective view of an implant according to another embodiment of the present invention.

FIG. 9 illustrates events that may be carried out in a process of producing a structure having trabecular bone-shaped bone ingrowth features, according to an embodiment of the present invention.

### DETAILED DESCRIPTION OF THE INVENTION

Before the present implants, surface features and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by claims that will be filed with the nonprovisional application claiming priority to this application.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a cavity” includes a plurality of such cavities and reference to “the surface” includes reference to one or more surfaces and equivalents thereof known to those skilled in the art, and so forth.

The publications discussed herein are provided solely for their disclosure prior to the filing date of the present application. The dates of publication provided may be different from the actual publication dates which may need to be independently confirmed.

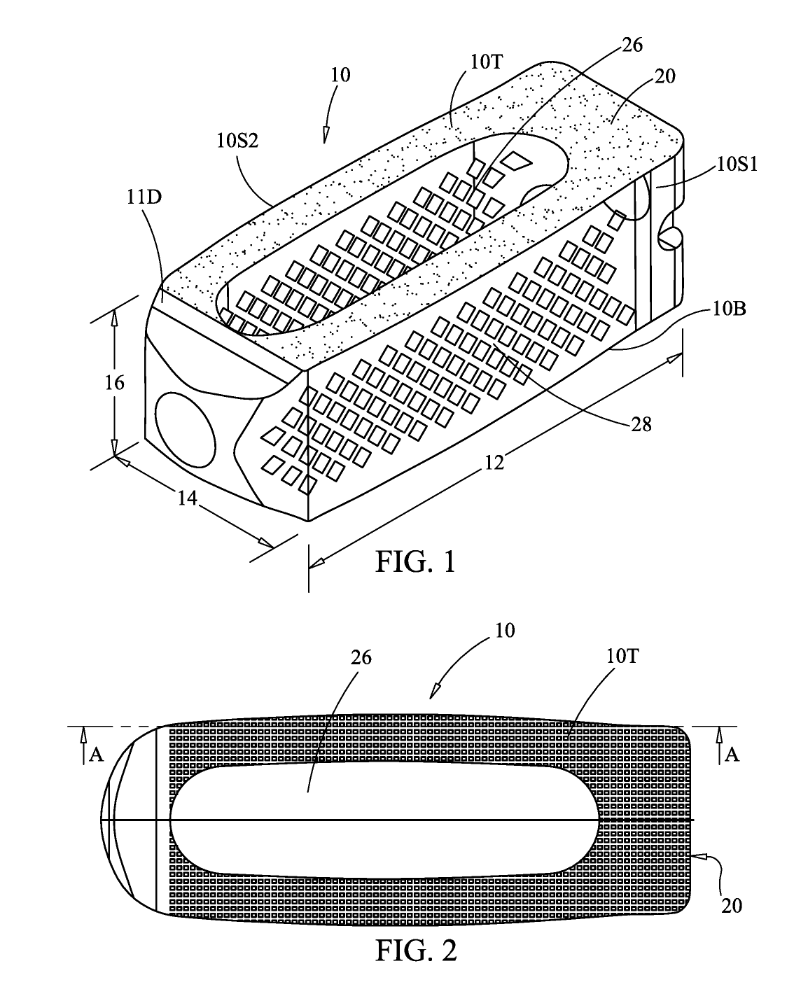

FIG. 1 shows a perspective view of an implant 10 according to an embodiment of the present invention. FIG. 2 shows a top view of the implant 10 of FIG. 1. Implant 10 is formed of a unitary body having a length dimension 12, width dimension 14 and height dimension 16. The body includes a top surface 10T and a bottom surface 10B extending along the length 12 of the implant 10 and also defining the width of the implant body. The top and bottom surfaces 10B, 10T may be mirror images of one another. First and second side surfaces 10S1 and 10S2 extend between the top 10T and bottom 10B surfaces on opposite sides of the implant 10 body.

The shape of the top 10T and/or bottom 10B surfaces can be curved or straight. When straight, they may have the same or different inclinations. When curved, they may have the same or different radii of curvature.

The first side 10S1 and second side 10S2 may have equal heights, or may be unequal. In one embodiment, first side 10S1 has a height that is substantially greater than a height of second side 10S2 giving the implant 10 a trapezoidal cross-sectional shape. In another embodiment the side heights are different but one or both of the top 10T and bottom 10B surfaces are curved. In another embodiment, the side heights are equal, giving the implant a rectangular or square cross section.

In at least one embodiment, the height of 10S1 is greater than the second height of 10S2 by a difference in the range of about 1.8 mm to about 2.2 mm. In at least one embodiment, the average height of the first side surface 10S1 over a length from a distal end to a proximal end of the implant 10 body is greater than the average height of the second side surface 10S2 over the length from the distal end 10D to the proximal end 10P. In at least one embodiment, the first height of 10S1, measured at any particular location along the length 12 of the first side 10S1 is greater than the height of the second side 10S2, measured at the same location along the length 12 on the second side 10S2. In at least one embodiment, each height difference between 10S1 and 10S2 at a same corresponding location along length 12 is in the range of about 1.8 mm to about 2.2 mm, typically about 2 mm. Thus, the first height of 10S1 is greater than the second height of 10S2 at all corresponding locations along the length of the implant body.

In the embodiment of FIG. 1, implant 10 is a substantially straight implant. However, in alternative embodiment, implant 10 could be curved. Examples of such curved configuration can be found, for example in U.S. Pat. No. 8,956,414, which is hereby incorporated herein, in its entirety, by reference thereto. Further descriptions of substantially straight implants can be found, for example, in U.S. Pat. No. 8,906,097, which is hereby incorporated herein, in its entirety, by reference thereto.

The top and bottom surfaces 10T, 10B are flat in the embodiment of FIG. 1, but may alternatively be convexly curved in a direction along the longitudinal axis L-L of the implant, which may better conform the top and bottom surfaces to the vertebrae forming the interbody disc space, as the vertebrae surfaces forming the interbody disc space are concave in the anterior-posterior direction, as well as the latero-medial direction. The convexity of the top and bottom surfaces 10T, 10B also results in reduced height of the distal and proximal portions relative to the height of the central portion on the same side of the implant 10. This condition is true for both sides 10S1, 10S2. The reduced height of the distal end and the tapered, varying height of the distal end portion 11D facilitate insertion of the implant 10 between adjacent vertebral bodies. The reduced height of the proximal end and tapered, varying height of the proximal end portion better conform this portion to the shape/contours of the inter-vertebral disk space for improved load sharing, that is with a more even load distribution over the length of the implant 10. Implants 10 can be manufactured to have a variety of sizes to accommodate different sizes of patients and different inter-vertebral locations. In one non-limiting example, implants 10 may be manufactured in lengths 12 of 22 mm, 24 mm, and 26 mm and in 1 mm height increments from 7 mm to 15 mm (each having the requisite height differential between heights of 10S1 and 10S2, or having equal heights). The width 14 may be about 9 mm or about 10 mm or in the range of about 9 mm to about 10 mm, although this may also vary.

Implant 10 is formed as a cage having a unitary body, with openings provided through the top and bottom surfaces 10T,10B to form cavity 26 (see FIG. 2), wherein the opening formed in the top surface 10T is in communication with the opening formed in the bottom surface 10B and is configured and dimensioned to receive graft material, such as bone particles or chips, demineralized bone matrix (DBM), paste, bone morphogenetic protein (BMP) substrates or any other bone graft expanders, or other substances designed to encourage bone ingrowth into the cavity 26 to facilitate the fusion. Although shown as a single, large cavity 26, implant 10 may be alternatively configured to provide two or more cavities that extend from top to bottom of the implant body 10 and through top and bottom surfaces 10T, 10B and provide the same function as cavity 26. Additionally implant 10 is provided with one or more side openings 28 as shown in FIG. 1. In the embodiment shown, the side openings 28 are provided through both sides 10S1, 10S2 and serve to reduce the stiffness of the implant body, as well as allow for additional bone ingrowth. In at least one embodiment, side openings are configured so as to reduce the stiffness below 350 KN/mm. In other embodiments, the stiffness value can be greater or smaller. Side openings 28 facilitate retention of the graft material in a honeycomb-like configuration and also encourage ingrowth of bone to form a honeycomb like capture of the implant 10. Further additionally or alternatively, at least one side opening 28 may function as an interface with a side impactor tool during lateral driving of the implant 10, as described in U.S. Pat. No. 8,906,097.

Implant 10 is preferably made from titanium, but can be made alternatively from PEEK (polyetheretherketone), Si₃N₄, or other metals, polymers or composites having suitable physical properties and biocompatibility.

Implant body 10 is provided with bone ingrowth features 20 on at least the top 10T and bottom 10B surfaces that encourage and facilitate bone ingrowth, fusion and/or mechanical locking of the implant 10 with surrounding bone. The surfaces 10T, 10B are preferably smooth, whether flat or curved, with the bone ingrowth features being formed into the surfaces. Several factors have shown their influence on bone ingrowth into porous implants, including porosity, duration of implantation, biocompatibility, implant stiffness and micro motion between the implant and adjacent bone. The bone ingrowth features 20 of the present invention not only allow and encourage bone ingrowth therein, but, because of their structure, form a “keying” or “locking” interface between the implant 10 and the adjacent bone. Thus, not only can fusion between the implant 10 and adjacent bone occur, but also mechanical interlocking of the implant 10 and the adjacent bone occurs. This provides for a stronger, more stable and longer lasting attachment between the implant 10 and adjacent bone.

Although the bone ingrowth features 20 are specifically described with regard to an interbody fusion implant 10, such as shown in FIG. 1, and can be used for transverse or transforaminal lumbar interbody fusion (TLIF), posterior lumbar interbody fusion (PLIF) or anterior lumbar interbody fusion, (ALIF), the bone ingrowth features 20 can be provided to any bone implant, including, but not limited to implants for use in the spine, knees, hips, shoulders, elbows, wrists, ankles fingers, toes, pelvis, cranium, long bones and other bone structures.

The bone ingrowth features 20 include cavities 22 that open to the surface of the structure that they are formed in. The opening 22P of the cavity 22 has a smaller cross sectional area than the cross sectional area of the body 22B of the cavity 22. That is, the body 22B of the cavity 22 is designed to be larger than the opening 22P. This allows bone ingrowth (osteoblast growth) through the opening 22P and into the body 22B. Typically, at least ten percent along the depth dimension 22D of the body 22B has a cross-sectional area that is greater than the cross-sectional area of the opening 22P, more typically at least twenty-five percent or at least fifty percent or at least sixty percent or at least seventy-five percent or at least ninety percent, or up to and including one hundred percent. Once bone growth has occurred in the body 22B it forms with a cross-sectional area that is larger than the cross-sectional area of the opening 22P. This results in a mechanical interlock of the implant and the bone (ingrown bone and bone adjacent the implant, which is integral with the ingrown bone). This key structure forming the mechanical interlock greatly strengthens the attachment of the implant 10 to the bone. Ideally the osteoblastic activity occurs such that the bone ingrowth fuses to the surfaces of the body 22B, but even if this does not occur, a mechanical interlock is formed.

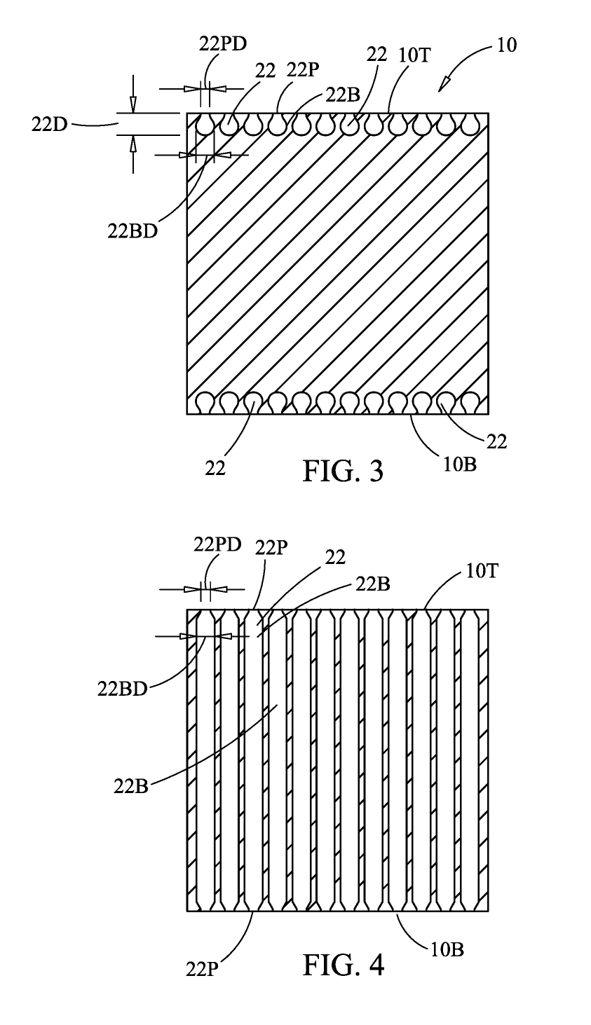

FIG. 3 is a partial, longitudinal sectional view of implant 10 taken along line A-A of FIG. 2, according to one embodiment of the present invention. In this embodiment bone ingrowth features 22 are bulbous or mushroom-shaped, with the features 22 in 10T appearing as inverted mushrooms and the features 22 in 10B appearing as upright mushrooms, with the stem of the mushroom or bulb opening 22P to the surface 10T, 10B and the body 22B of the mushroom or bulb extending into the implant 10. In this embodiment, both cross-sectional areas of the opening 22P and the body 22B are circular. In FIG. 3, the diameter 22PD of the opening 22P has a value in the range of from about 50 μm to about 1 mm, preferably from about 50 μm to about 600 μm and the diameter 22BD of the body (largest cross sectional diameter) 22B has a value in the range of from about 100 μm to about 1.2 mm, where, of course, the diameter 22BD in each embodiment is larger than the diameter 22BP. Although the sizes of the openings 22P and the bodies 22B are illustrated as all being equal in the embodiments shown herein, it is noted that either or both of the sizes of the openings 22P and bodies 22B may be varied, within the ranges provided, so as to be unequal from each other, as formed in an implant. Variations in the sizes can be used to further fine tune the stiffness characteristics of the implant body 10 and/or to enhance osteoblast activity.

The depth 22D of the bone ingrowth features 22 (i.e., the distance that the features 22 extend into the implant 10, measured from the surface of the implant 10) may be a value in the range of from about 250 μm, up to half the height 16 of the implant 10.

FIG. 4 is a partial, longitudinal sectional view of implant 10 taken along line A-A of FIG. 2, according to another embodiment of the present invention. In this embodiment, the bone ingrowth features 22 extend all the way through the implant 10 (along the height 16 dimension, as shown, although these type of features 22 may extend through an implant along any dimensional direction). The features 22 are similar to those in FIG. 3, if extended through the body of the implant 10 so that the body 22B of a top feature 22 opens to the body 22B of a bottom feature 22. Thus, the bone ingrowth features 22 of FIG. 4 include two openings 22P, one at the top surface 10T and one at the bottom surface 10B of the implant 10. A single body 22B extends through the implant and communicates with the openings 10P at the top 10T and bottom 10B surfaces of the implant 10. Openings 22P in FIG. 4 are circular and taper to the main portion of body 10B, which is cylindrical, with a circular cross-section. Dimensions 22PD and 22PB are the same as for those provided with regard to FIG. 3.

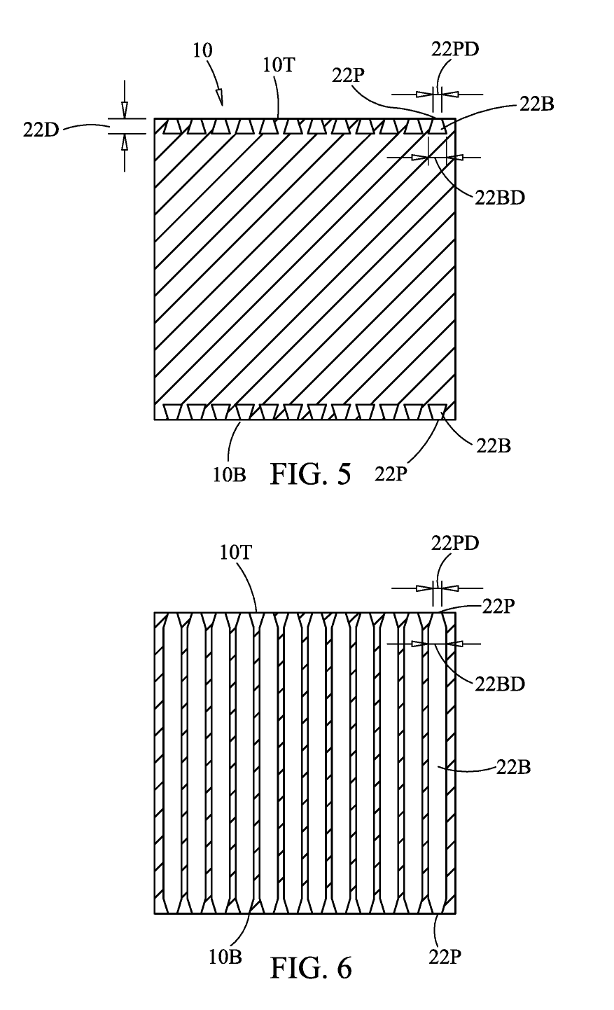

FIG. 5 is a partial, longitudinal sectional view of implant 10 taken along line A-A of FIG. 2, according to another embodiment of the present invention. In this embodiment bone ingrowth features 22 are conical, with the small end of the cone shape forming the opening 22P of the feature 22. Thus in this embodiment, one hundred percent of the body 22B along the depth dimension 22D of the body 22B has a cross-sectional area that is greater than the cross-sectional area of the opening 22P. In this embodiment, both cross-sectional areas of the opening 22P and the body 22B are circular. In FIG. 5, the diameter 22PD of the opening 22P has a value in the range of from about 100 μm to about 1 mm and the diameter 22BD of the body (largest cross sectional diameter) 22B has a value in the range of from about 100 μm to about 1.2 mm, where, of course, the diameter 22BD in each embodiment is larger than the diameter 22BP. Although the sizes of the openings 22P and the bodies 22B are illustrated as all being equal in the embodiments shown herein, it is noted that either or both of the sizes of the openings 22P and bodies 22B may be varied, within the ranges provided, so as to be unequal from each other, as formed in an implant.

The depth 22D of the bone ingrowth features 22 (i.e., the distance that the features 22 extend into the implant 10, measured from the surface of the implant 10) may be a value in the range of from about 250 μm, up to half the height 16 of the implant 10.

FIG. 6 is a partial, longitudinal sectional view of implant 10 taken along line A-A of FIG. 2, according to another embodiment of the present invention. In this embodiment, the bone ingrowth features 22 extend all the way through the implant 10 (along the height 16 dimension, as shown, although these type of features 22 may extend through an implant along any dimensional direction). The features 22 are similar to those in FIG. 5, if extended through the body of the implant 10 so that the body 22B of a top feature 22 opens to the body 22B of a bottom feature 22. Thus, the bone ingrowth features 22 of FIG. 6 include two openings 22P, one at the top surface 10T and one at the bottom surface 10B of the implant 10. A single body 22B extends through the implant and communicates with the openings 10P at the top 10T and bottom 10B surfaces of the implant 10. Openings 22P in FIG. 4 are circular and taper to the main portion of body 10B, which is cylindrical, with a circular cross-section. Dimensions 22PD and 22PB are the same as for those provided with regard to FIG. 3. The percentage of the surface area of surfaces 10T, 10B that are taken up by the openings 22P may vary, but are typically configured to provide a porosity having a value in the range of from about 40% to about 80%. The openings are typically regularly spaced, but need not be.

Although all embodiments of bone ingrowth features 22 specifically described above have circular openings 22P and bodies 22B having circular cross-sectional areas, the present invention is not limited to these shapes, as opening 22P could have any shape, including, but not limited to oval, elliptical, polygonal or irregular. Likewise, a portion or all of body 228 may have a cross-sectional shape that is not circular, including, but not limited to oval, elliptical, polygonal or irregular.

Implants 10 containing bone ingrowth features 22 or layers containing surface features 22 that can be fixed to an implant can be made by 3D printing, direct metal laser sintering (DMLS), selective laser melting (SLM), electron beam melting (EBM), laser engineered net shaping (LENS), or the like.

FIG. 8 shows a perspective view of an implant 10 according to another embodiment of the present invention. The embodiment of FIG. 8 can have any or all of the same features as the embodiment of FIG. 1, with the only difference being that of the bone ingrowth features 20′ that are provided with the embodiment of FIG. 8. In the embodiment of FIG. 8, the bone ingrowth features 20′ are features are formed and shaped like trabecular bone structure as captured by micro-CT scanning for example.

Bone ingrowth features 20′ may be provided on at least the top 10T and bottom 10B surfaces that encourage and facilitate bone ingrowth, fusion and/or mechanical locking of the implant 10 with surrounding bone. The surfaces 10T, 10B are preferably smooth, whether flat or curved, with the bone ingrowth features being formed into the surfaces.

Although the bone ingrowth features 20′ are specifically described with regard to an interbody fusion implant 10, such as shown in FIG. 8, and can be used for transverse or transforaminal lumbar interbody fusion (TLIF), posterior lumbar interbody fusion (PLIF) or anterior lumbar interbody fusion, (ALIF), the bone ingrowth features 20′ can be provided to any bone implant, including, but not limited to implants for use in the spine, knees, hips, shoulders, elbows, wrists, ankles fingers, toes, pelvis, cranium, long bones and other bone structures.

The bone ingrowth features 20′ are shown more clearly in the magnified portion of top surface 10T shown in the inset view of FIG. 8. The bone ingrowth features include features analogous to the features of trabecular bone, including trabeculae 23 and openings 25 that would contain bone marrow and blood vessels in the trabecular bone. Openings 25 include cavities 22 that open to the surface of the structure that they are formed in. At least some, typically at least a majority up to all, of the openings 25 have a smaller cross sectional area than the cross sectional area of the cavities 25C that they open to. This allows bone ingrowth (osteoblast growth) through the opening 25 and into the cavity 25C with the formation of secondary osteonal structures inside the cavities 25C.

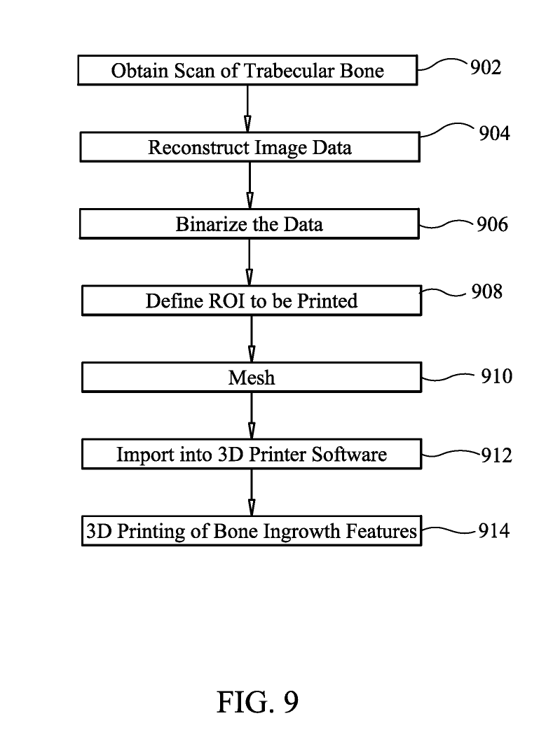

The trabecular bone-shaped bone ingrowth features 20′ may be produced by three-dimensional (3D) printing techniques. FIG. 9 illustrates events that may be carried out in a process of producing a structure having the trabecular bone-shaped bone ingrowth features 20′. At event 902, one or more scans of trabecular bone are obtained to provide digital images of the lattice structure of the trabecular bone. The scan(s) obtained may be from scanning using micro-computerized tomography (micro-CT) apparatus, for example. Healthy (e.g., non-osteoporotic) vertebral cancellous bone is typically used as the subject of the scan(s). Examples of micro-CT apparatus that may be used include, but are not limited to: Siemens (Inveon CT); CT imaging (Tomoscope Synergy); or Scanco Medical (XtremeCT). Preferably a standard micro-CT scanning process is performed with maximum intensity projection of the reconstructed slices. Maximum intensity projection (MIP) is a volume rendering method for 3D data that projects in the visualization plane the voxels with maximum intensity to maximize contrast. MIP enhances the 3D nature of certain scanned objects relative to the adjacent structures

The data obtained from the scanning in event 902 is then processed to reconstruct the image data of the scanned trabecular bone at event 904. At event 906, the image data is binarized. If the resolution of the scan is higher than required for the bone ingrowth features 20′ to be printed, the dataset can be resized. Thresholding is then carried out as usual. Image filters can be useful when thresholding. At event 908, a region of interest (ROI) is selected/defined as the portion of the image to be reproduced when printing the bone ingrowth features 20′.

At event 910 meshing is performed. A 3D model representing the surface of the binary object is constructed. This meshing procedure typically comprises used of polygonal elements of which the vertices and normals are saved. Data outputs in commonly used 3D file types, including, but are not necessarily limited to: .stl and .ply. A check is performed for which file type is best for the 3D printer to be used. Surface rendering of the micro-CT model can be performed, for example, using Bruker CTVol software.

At event 912, the meshed computer model resulting from event 910 is imported into the 3D printer software and rescaled to the size required to perform the 3D printing of the bone ingrowth features 20′, in preparation for 3D printing of the lattice structure. Various types of 3D printing methodologies may be used for the 3D printing, including, but not limited to, direct metal laser sintering (DMLS) or vapor deposition type 3D printing. At event 914, the bone ingrowth features 20′ are produced layer-by-layer, using the meshed model to map the locations of the structures in each layer that are printed and built up on one another, layer-by-layer, to produce a replica of the three-dimensional lattice structure of the trabecular bone that was scanned. The features 20′ are produced on a surface, which may be a surface of any of the bone implant structures mentions previously, or any surface into which bone ingrowth is desired. Features 20′ may be made of any of the materials described herein with regard to other embodiments.

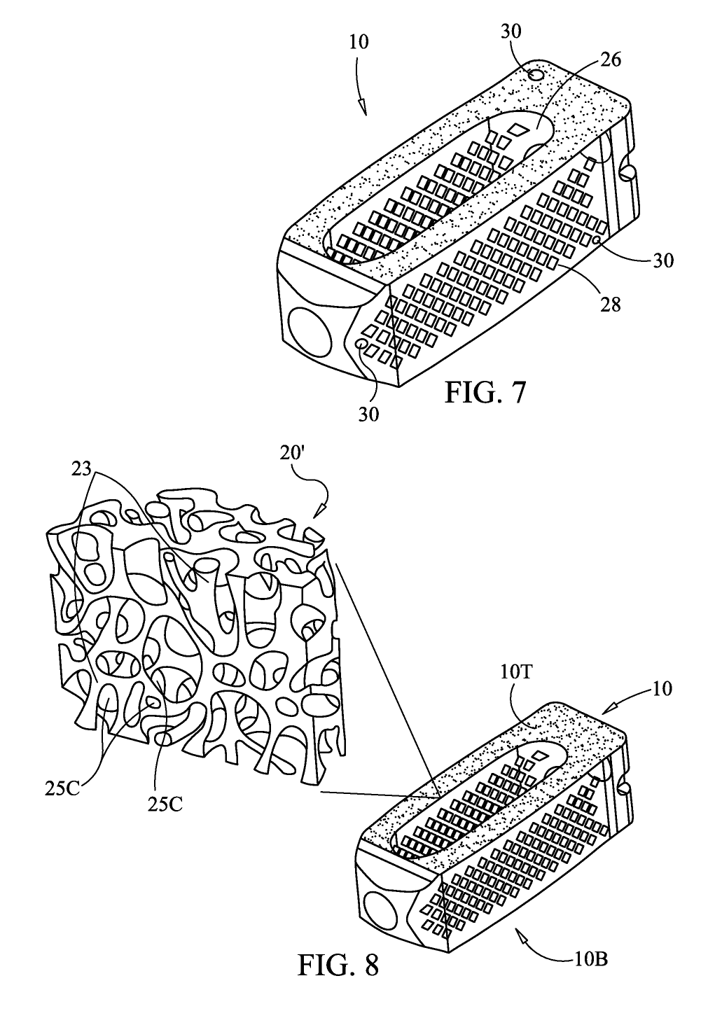

When implant 10 is made from PEEK, carbon-filled PEEK, or any other radiolucent material, the implant 10 may optionally be provided with one or more (typically at least three) radiopaque markers 30 to facilitate visualization of the implant 10 during the procedure, so as to confirm that the implant is being delivered along a desirable delivery pathway and that the implant 10 is maintaining a desirable orientation. In the example shown in FIG. 7, one marker 30 is provided adjacent side 10S1 at or near the top surface 10T of the proximal end portion (FIG. 1A), a second marker 30 is provided adjacent side 10S2 at or near the bottom surface 10B of the proximal end portion and a third marker 30 is provided horizontally, adjacent the distal end portion in a location 30′ (FIG. 1C) between sides 10S1 and 10S2. By placing radiopaque markers 30 as described, this enables radiographic viewing of the markers 30, at any location along the delivery pathway and during the procedure, as well as post-procedurally, to accurately determine the three-dimensional positioning of the implant 10. Thus, not only can the radiographic imaging determine the location that the implant 10 is placed in, it can also determine the three-dimensional orientation of the implant relative to the anatomy at the location that it is placed in.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention.

That which is claimed is:

1. A surgical implant comprising:
a main body having top, bottom and side surfaces; and
bone ingrowth features formed in a least one of said top, bottom and side surfaces;
wherein each of said bone ingrowth features comprises an opening that opens to said at least one of said top, bottom and side surfaces, and a body that extends from said opening into said implant;
wherein said opening has a first cross-sectional dimension and said body has a second cross-sectional dimension; and
wherein said second cross-sectional dimension is greater than said first cross-sectional dimension.

2. The surgical implant of claim 1 wherein said opening has a first cross sectional area and said body has a second cross-sectional area; and
wherein said second cross-sectional area is greater than said first cross-sectional area.

3. The surgical implant of claim 1, wherein said side surfaces are smooth.

4. The surgical implant of claim 1, wherein said bone ingrowth features are mushroom-shaped.

5. The surgical implant of claim 1, wherein said bone ingrowth features are conical-shaped.

6. The surgical implant of claim 1, wherein said bone ingrowth features are formed and shaped like trabecular bone structure.

7. The surgical implant of claim 6, wherein said bone ingrowth features are produced by 3D printing from a scanned image of trabecular bone.

8. The surgical implant of claim 1, wherein said opening has a first diameter and said body has a second diameter, said second diameter being greater than said first diameter.

9. The surgical implant of claim 8, wherein said first diameter comprises a value in a range from about 50 μm to about 600 μm and said second diameter comprises a value in a range from about 100 μm to about 1.2 mm.

10. The surgical implant of claim 1, wherein said main body comprises titanium.

11. The surgical implant of claim 1, wherein said main body comprises PEEK.

12. The surgical implant of claim 1, wherein said surgical implant comprises an interbody fusion implant.

13. The surgical implant of claim 1 produced by 3D printing.

14. The surgical implant of claim 1 produced by direct metal laser sintering.

15. A structure for facilitating bone attachment comprising:
a structure comprising a surface; and
bone ingrowth features formed in said structure;
wherein said bone ingrowth features comprise openings that open to said surface, and bodies that extend from said openings into said structure;
wherein said openings have first cross-sectional dimensions and said bodies have second cross-sectional dimensions; and
wherein at least one of said second cross-sectional dimensions is greater than at least one of said first cross-sectional dimensions from which said bodies extend, respectively.

16. The structure of claim 15, wherein at least one of said openings has a first cross sectional area and at least one of said bodies that extends from said at least one of said openings, respectively, has a second cross-sectional area; and
wherein said second cross-sectional area is greater than said first cross-sectional area.

17. The structure of claim 15, wherein said surface is smooth.

18. The structure of claim 15, wherein said bone ingrowth features are mushroom-shaped.

19. The structure of claim 15, wherein said bone ingrowth features are conical-shaped.

20. The structure of claim 15, wherein at least one of said openings has a first diameter and at least one of said bodies that extends from said at least one of said openings, respectively, has a second diameter, said second diameter being greater than said first diameter.

21. The structure of claim 20, wherein said first diameter comprises a value in a range from about 50 μm to about 600 μm and said second diameter comprises a value in a range from about 100 μm to about 1.2 mm.

22. The structure of claim 15 produced by 3D printing.

23. The structure of claim 15 produced by direct metal laser sintering.

24. A structure for facilitating bone attachment comprising:
a structure comprising a surface; and
bone ingrowth features formed in said structure;
wherein said bone ingrowth features are formed and shaped like trabecular bone structure; and
wherein said bone ingrowth features are produced by 3D printing from a scanned image of trabecular bone.

25. The structure of claim 24, wherein at least one of said bone ingrowth features comprises an opening that opens to said surface, and a body that extends from said opening into said structure;
wherein said opening has a first cross-sectional dimension and said body has a second cross-sectional dimension; and
wherein said second cross-sectional dimension is greater than said first cross-sectional dimension.

26. A method of making a structure for facilitating bone attachment, said method comprising:
obtaining a scan of trabecular bone to provide an image of lattice structure of the trabecular bone;
processing the scan to form a computer image model of the lattice structure; and
forming said lattice structure on a surface, using a 3D printing technique, said forming performed layer-by-layer to reproduce a 3D structure of the lattice structure of the trabecular bone.

27. The method of claim 26, wherein said scan is performed by using a micro-computer tomography (CT) scanner.

28. The method of claim 26, wherein said 3D structure comprises titanium.

29. The method of claim 26, wherein said 3D structure comprises PEEK.
